SEQUENCE LISTING (21 sequences)

SEQ ID NO: 1 - DNA, 2049 bp, Solanum melongena
  source 1..2049: mol_type = genomic DNA; note = Nucleotide sequence of the SmABCG2 gene
atggagatagaggttacaagtggtagtagtgatattgagaaaggtgtaatgcatagacaaggagttgcatatttggtatgggaagatttaacagtaatgttgccaaattttggtcagggaccaactaagaagttgttacatggactaagtggttatgctgaacctggtagaattatggctattatgggtccttctggttctggaaaatctacacttcttgatactttagcaggtaggctgtcgacgaatgttgtgatgactggaaatatccttctaaatgggaagaagaggaggttggactatggtgttgttgcttatgttactcaagaggatacactgctgggaactctaacacctagagagacaattacttattcagcccatcttcgacttccaactagcatgacgaaagaagaggtaaacgatattgtggagggaacaataatggaaatggggctaggggattgtgctgatcgggtggtaggaaattggcaagtaagaggaataagtggtggcgagaaaaagagactaagcattgcacttgaaatccttgtacgacctcgtatactttttcttgatgaacctaccactggtcttgacagtgcctcagcattctttgttgttcaagctcttaaaaatatctcccgcgatggaagaacagttatctcatccattcatcagcctagtagtgaagttttcgcgttgtttgatgacctctttctgttgtctggtggagagactgtatattttggagaagcaaaattggcagtacagttctttgctgaatcgggatttccatgtccaagtaggagaaatccatcagatcatttcctacgttgtgttaattcagactttgacgttgtcacagccacattgaaaggctcacaaagactccgggaaacacataaaagtgactacttaatgaacatggcaacagcagaaataaaagaaatgcttgccagcaaatataaacgctcagcatatgcaacgagggccaggtcacgtatgcgagaactcttggccactcaaggtgttgagattgaaacagtaaaaggaagtcaagctggttggggaaagcaacttttgacactgacgcggagatcatttgtaaacatgtcaagagacaagggatattattggtctcgtatagtaatatatatcattgtagcttttgccgttggtaccctcttctatgatgttggcaccagttacacagcaatcttagctcgtggagcttgtggtgggtttgtcacgggctacatgactttcatgtccattggcggctttccatctttcatagaagaaatgaaggtttttactaaagaaaggcttaacgggcactatggtgttggagctttcatattggcaaacttcctctcatcatttccattcttagttgcagtttcactcatcactgggaccatcacttattacatggtgtttcgggctagattcttccgttatgtgtttttctgcttaaacctttttggttgtatcgctgtcgttgagagctgcatgatgattgtagcttcacttgttccaaacttcttaatgggaatcattacaggagctggtgtgcttggaatcatgatgatgactgctggatttttccgtttgctgcctgatcttcccaagccaatttggcgctatccaatttcatttattgggtatggggcatggggtttacagggatcatacaaaaatgacatgattggacttgtattcgatccactaattcctggcggagagaaactaaaaggggaagatgtgatcacgaacatgttcaaattgtcgttggatcactccaaatggtgggatttacttgcgttatactccctcatcgtcatgtacaggcttctattctttataattctcaagctaaaagagcgagctacaccatttttccgatcaatgtatgctaagagaacgatgcatcaactcaaaaggcgtccttcattcaagaggaaaccatctttatcctattcaaaaaggcaacatactctcaggtcattgtcttctcaagaaggtcttagttctccaatcccatag

SEQ ID NO: 2 - DNA, 416 bp, Solanum melongena
  source 1..416: mol_type = genomic DNA; note = Sequence of the first exon region of the SmABCG2 gene
atggagatagaggttacaagtggtagtagtgatattgagaaaggtgtaatgcatagacaaggagttgcatatttggtatgggaagatttaacagtaatgttgccaaattttggtcagggaccaactaagaagttgttacatggactaagtggttatgctgaacctggtagaattatggctattatgggtccttctggttctggaaaatctacacttcttgatactttagcaggttagtctttgattatgaatactttggactatgtccgattagatagatgaaaagaaattatctagcatttaaaccctagtcgattgtcaggaaatagcctctctacctctgtaaagtagtggtaaggtctgcgtacatttgtgagatttcactggtatgttgttgttagttaaatcctagtcga

SEQ ID NO: 3 - DNA, 25 bp, synthetic construct
  source 1..25: mol_type = other DNA; note = Sequence of forward primer 26pF
tgtcccaggattagaatgattaggc

SEQ ID NO: 4 - DNA, 25 bp, synthetic construct
  source 1..25: mol_type = other DNA; note = Sequence of reverse primer 29pR
agccctcttctttcgatccatcaac

SEQ ID NO: 5 - DNA, 20 bp, synthetic construct
  source 1..20: mol_type = other DNA; note = Sequence of sgRNA for editing SmABCG2 gene
aagttgttacatggactaag

SEQ ID NO: 6 - DNA, 20 bp, synthetic construct
  source 1..20: mol_type = other DNA; note = Another sequence of sgRNA for editing SmABCG2 gene
tcctcttcttcccatttaga

SEQ ID NO: 7 - DNA, 42 bp, synthetic construct
  source 1..42: mol_type = other DNA; note = Forward primer KO1F0 for target site 1
tgaagttgttacatggactaaggttttagagctagaaatagc

SEQ ID NO: 8 - DNA, 44 bp, synthetic construct
  source 1..44: mol_type = other DNA; note = Reverse primer KO2R0 for target site 1
aactcctcttcttcccatttagacaatctcttagtcgactctac

SEQ ID NO: 9 - DNA, 40 bp, synthetic construct
  source 1..40: mol_type = other DNA; note = Forward primer KO1F for target site 2
aataatggtctctattgaagttgttacatggactaaggtt

SEQ ID NO: 10 - DNA, 37 bp, synthetic construct
  source 1..37: mol_type = other DNA; note = Reverse primer KO2R for target site 2
attattggtctctaaactcctcttcttcccatttaga

SEQ ID NO: 11 - DNA, 20 bp, synthetic construct
  source 1..20: mol_type = other DNA; note = Specific primer Cas9-F designed according to Cas9 in vector pKSE401
acgacgatgacctggataac

SEQ ID NO: 12 - DNA, 21 bp, synthetic construct
  source 1..21: mol_type = other DNA; note = Specific primer Cas9-R designed according to Cas9 in vector pKSE401
accttgtcatcgaagagatgg

SEQ ID NO: 13 - DNA, 20 bp, synthetic construct
  source 1..20: mol_type = other DNA; note = Detection primer pair G2-F1 used to detect the editing profile at target 1
tagtgatattgagaaaggtg

SEQ ID NO: 14 - DNA, 18 bp, synthetic construct
  source 1..18: mol_type = other DNA; note = Detection primer pair G2-R1 used to detect the editing profile at target 1
gaatagaagaaatggacc

SEQ ID NO: 15 - DNA, 22 bp, synthetic construct
  source 1..22: mol_type = other DNA; note = Detection primer pair G2-F2 used to detect the editing profile at target 2
ttcaatgagtaatttgcctctg

SEQ ID NO: 16 - DNA, 18 bp, synthetic construct
  source 1..18: mol_type = other DNA; note = Detection primer pair G2-R2 used to detect the editing profile at target 2
cacccgatcagcacaatc

SEQ ID NO: 17 - DNA, 26 bp, Solanum melongena
  misc_feature 1..26: note = the original sequencing signal indicating target 1 for gene editing of SmABCG1 in eggplant
  source 1..26: mol_type = other DNA
aagaagttgttacatggactaagtgg

SEQ ID NO: 18 - DNA, 24 bp, synthetic construct
  misc_feature 1..24: note = the original sequencing signal indicating target 1 of SmABCG1 after being edited
  source 1..24: mol_type = other DNA
aagttgttacatggacttaagtgg

SEQ ID NO: 19 - DNA, 43 bp, synthetic construct
  misc_feature 1..43: note = the DNA sequence of target 1 after being edited
  source 1..43: mol_type = other DNA
accaactaagaagttgttacatggacttaagtggttatgctga

SEQ ID NO: 20 - DNA, 43 bp, synthetic construct
  misc_feature 1..43: note = complementary sequcence of SED ID NO: 19
  source 1..43: mol_type = other DNA
tggttgattcttcaacaatgtacctgaattcaccaatacgact

SEQ ID NO: 21 - protein, 13 aa, synthetic construct
  REGION 1..13: note = amino acid sequence encoded by the edited target 1 of SmABCG1
PTKKLLHGLKWLC

(19) United States
(12) Patent Application Publication
(10) Pub. No.: US 2026/0043044 A1
(43) Pub. Date: Feb. 12, 2026

(54) USE OF SmABCG2 GENE IN CULTIVATION OF MALE STERILE LINES IN EGGPLANT

(71) Applicants: ZHENGZHOU UNIVERSITY, Zhengzhou City (CN); ZAAS, Zhumadian Academy of Agricultural Sciences, Zhumadian City (CN)

(72) Inventors: Yanjie ZHANG, Zhengzhou City (CN); Yong WANG, Zhengzhou City (CN); Qiu YANG, Zhengzhou City (CN); Yan LI, Zhengzhou City (CN); Jiansen FANG, Zhengzhou City (CN); Jun JIANG, Zhengzhou City (CN)

(21) Appl. No.: 19/033,097

(22) Filed: Jan. 21, 2025

(30) Foreign Application Priority Data
Aug. 12, 2024 (CN) 202411102742.5

(51) Int. Cl. C12N 15/82; C12N 9/22; C12N 15/11; C12Q 1/6895

(52) U.S. Cl. CPC C12N 15/8289; C12N 9/22; C12N 15/11; C12N 15/8205; C12N 15/8213; C12Q 1/6895; C12N 2310/20; C12Q 2600/13

(57) ABSTRACT

The use of SmABCG2 gene in the cultivation of male sterile line in eggplant is provided, belonging to the field of genetic engineering. In the present disclosure, on the basis of CRISPR/Cas9 technology, the SmABCG2 gene is edited using an sgRNA having a nucleotide sequence as shown in SEQ ID NO: 5, or a combination of sgRNAs as shown in SEQ ID NOs. 5 and 6, to knock out the SmABCG2 gene and obtain male sterile lines in eggplant. Due to the stable sterility of these eggplant lines in the northern semi-arid region of China, male sterile lines with different genetic backgrounds could be created, and the male sterile lines can be applied to crossbreeding and seed production.

### CROSS-REFERENCE TO RELATED APPLICATION

This patent application claims the benefit and priority of Chinese Patent Application No. 202411102742.5 filed with the China National Intellectual Property Administration on Aug. 12, 2024, the disclosure of which is incorporated by reference herein in its entirety as part of the present application.

### REFERENCE TO SEQUENCE LISTING

A computer readable XML file entitled “GWP20240806196-sequence listing”, which was created on Dec. 4, 2024 with a file size of about 27,521 bytes, contains the sequence listing for this application, has been filed with this application, and is hereby incorporated by reference in its entirety.

### TECHNICAL FIELD

The present disclosure belongs to the technical field of genetic engineering, and in particular, relates to the use of SmABCG2 gene in the cultivation of male sterile lines in eggplant.

### BACKGROUND

Eggplant (Solanum melongena L.) is a solanaceous vegetable that is widely grown and consumed around the world, originating in the subtropical regions of Asia, and China is considered to be one of the origins of eggplant. Eggplant is a self-pollinating crop with heterosis. The use of the heterosis can greatly improve the yield, disease resistance, and stress resistance of eggplant. At present, the production of eggplant hybrids is still dominated by the manual emasculation and pollination that is time-consuming and laborious, resulting in high seed production cost and unguaranteed seed purity. The introduction of male sterile lines in seed production can optimize the seed production procedure, reduce labor costs, improve the purity of hybrid seeds, and avoid the dispersal of parents. Therefore, eggplant crossbreeding studies are mainly focused on the development of male sterile lines that are more convenient for production and application.

ATP-binding cassette transporter (ABC)G family transporters are involved in the transport of lipids synthesized in anther tapetum. Cytological studies showed that ABCG15 and ABCG26 synergistically control the transfer of lipids within the anther in the plant, where ABCG26 mainly transports lipid from tapetum cells to the cell layer of the anther wall, and ABCG15 mainly transports lipid from the tapetum to the anther sac. The lipid molecules finally deposit outside the cell wall. As an advanced biotechnology, gene editing provides unique advantages for the creation of male sterile materials. Typically, male sterility is caused by genetic defects or mutation, and traditional breeding methods often fail to accurately repair these genetic variations, resulting in difficulties in breeding. Gene editing technology can accurately repair and adjust genetic variations, and then create male sterile germplasm with excellent traits.

### SUMMARY

A first objective of the present disclosure is to provide methods for using the SmABCG2 gene in the cultivation of eggplant male sterile lines, where the SmABCG2 gene has the nucleotide sequence as shown in SEQ ID NO: 1.

A second objective of the present disclosure is to provide a single guide RNA (“sgRNA”) that edits the SmABCG2 gene, where the sgRNA has the nucleotide sequence shown in SEQ ID NO: 5.

A third objective of the present disclosure is to provide an sgRNA combination that edits the SmABCG2 gene, where the sgRNA combination includes two sgRNAs having the nucleotide sequences as shown in SEQ ID NO: 5 and SEQ ID NO: 6, respectively.

A fourth objective of the present disclosure is to provide the use of an engineered biological material including the sgRNA or the sgRNA combination mentioned above for the creation of a gene edited plant.

A fifth objective of the present disclosure is to provide method for using the sgRNA or the sgRNA combination in the site-directed mutagenesis of a gene in eggplant and/or in the cultivation of male sterile lines in eggplant.

A sixth objective of the present disclosure is to provide an engineered Agrobacterium tumefaciens line or a recombinant vector, where the engineered bacterium or the recombinant vector contains the sgRNA or the sgRNA combination.

A seventh objective of the present disclosure is to provide methods for using the engineered bacterium or the recombinant vector in the site-directed mutagenesis of a gene in eggplant and/or in the cultivation of eggplant male sterile materials.

An eighth objective of the present disclosure is to provide a CRISPR/Cas9 editing system that includes the sgRNA or the sgRNA combination.

A ninth objective of the present disclosure is to provide a method of creating an SmABCG2 gene knockout mutant in eggplant using a CRISPR/Cas9 editing system, where the method includes the steps of:
(1) synthesizing the sgRNA or the sgRNA combination;
(2) ligating the sgRNA into pKSE401 to obtain pKSE401-G1; or ligating the sgRNA combination into the pKSE401 to obtain pKSE401-G2;
(3) transforming the pKSE401-G1 or the pKSE401-G2 into Agrobacterium tumefaciens to obtain recombinant Agrobacterium tumefaciens lines, performing Agrobacterium-mediated transformation of eggplant, and then screening positive plants.

Advantageously, an eggplant having a gene knockout of SmABCG2 is male sterile.

Compared with the conventional art, the present disclosure has the following beneficial effects:

In the present disclosure, SmABCG2 gene is knocked out using CRISPR/Cas9 technology to obtain the male sterile lines of eggplant. Due to the stable male sterility of these eggplant lines grown in the northern semi-arid region of China, male sterile lines can be created in eggplant of different genetic backgrounds, thereby being applied to crossbreeding and seed production.

### BRIEF DESCRIPTION OF THE DRAWINGS

FIG. 1 shows agarose gel electrophoresis results of PCR amplification of genomic DNA from wild-type and TO plants using Cas9-F and Cas9-R primers, indicating that the TO generation plant has a Cas9 gene fragment.

FIG. 2 shows a sequencing peak diagram of base sequences at the gene editing sites in Example 4. The sequences in FIG. 2 are as follows:

AAGAAGTTGTTACATGGACTAAGTGG
(SEQ ID NO: 17; the original sequencing
signal indicating target 1 for gene editing of SmABCG1 in eggplant);
AAGTTGTTACATGGACTTAAGTGG
(SEQ ID NO: 18; the original sequencing signal
indicating target 1 of SmABCG1 after being edited);
ACCAACTAAGAAGTTGTTACATGGACTTAAGTGGTTATGCTGA
(SEQ ID NO: 19; the DNA sequence of target 1 after being edited);
TGGTTGATTCTTCAACAATGTACCTGAATTCACCAATACGACT
(SEQ ID NO: 20; complementary sequence of SED ID NO: 19);
PTKKLLHGLKWLC
(SEQ ID NO: 21; amino acid sequence encoded by the edited target 1
of SmABCG1).

FIG. 3 shows seed abortion in an abcg2 mutant.

FIG. 4A: shows a comparison of pollen germination rates between abcg2 mutant and wild-type plants; FIG. 4B shows pollen germination rates and pollen micromorphologies of wild-type and abcg2 mutant plants; and FIG. 4C shows pollen micromorphologies of an abcg2 mutant.

### DETAILED DESCRIPTION OF THE EMBODIMENTS

The eggplant variety used in the examples of the present disclosure was “Solanum melongena L cv. Sanyueqie”, which was donated by Tian Shibing, a researcher of Chongqing Academy of Agricultural Sciences. The binary vector pKSE401, intermediate vector pCBC-DT1T2, Escherichia coli DH5α and Agrobacterium tumefaciens LBA4404 used in the examples of the present disclosure were obtained from commercial suppliers and stored in the laboratory; the genomic DNA extraction kit was purchased from TIANGEN Biotech (Beijing) Co., Ltd.; the DNA Ligation Kit Ver. 2.0 (T4 DNA ligase/Solution I), restriction enzymes, Taq DNA polymerase, PrimeSTAR HS DNA high-fidelity polymerase were all products of TaKaRa Biotechnology (Dalian) Co., Ltd.; the DNA purification kit (spin-column) was the product of Nanjing Vazyme Biotech Co., Ltd.; and the remaining reagents were all analytically pure reagents, wherein agar, rifampicin, streptomycin, YEB solid medium, kanamycin, LB solid medium, pollen nutrient liquid, sucrose, MS salt solution, indoleacetic acid, zeatin and carbenicillin were all purchased from Sangon Biotech (Shanghai) Co., Ltd., and Murashige & Skoog Basal Medium with Vitamins (MS medium) was purchased from PhytoTech Labs.

### Example 1 Design of Target Sites and Primers

The coding region of SmABCG2, the gene that controls fertility in eggplant, was submitted to the online website (crispor.tefor.net). The genomic sequence of eggplant was selected, the PAM sequence was NGG, and the sequence that spans across introns was avoided. After the submission, the target sites that have a high score and a coding location near the 5′ end were selected, and then submitted to the website (www.rgenome.net/cas-offinder) for off-target detection. To ensure the editing efficiency, two sgRNA sequences were finally selected for SmABCG2.

The coding sequence of the SmABCG2 gene is shown in SEQ ID NO: 1:
(SEQ ID NO: 1 (CDs))
ATGGAGATAGAGGTTACAAGTGGTAGTAGTGATATTGAGAAAGGTGTAATGCA
TAGACAAGGAGTTGCATATTTGGTATGGGAAGATTTAACAGTAATGTTGCCAAATTTT
GGTCAGGGACCAACTAAGAAGTTGTTACATGGACTAAGTGGTTATGCTGAACCTGGT
AGAATTATGGCTATTATGGGTCCTTCTGGTTCTGGAAAATCTACACTTCTTGATACTTTA
GCAGGTAGGCTGTCGACGAATGTTGTGATGACTGGAAATATCCTTCTAAATGGGAAG
AAGAGGAGGTTGGACTATGGTGTTGTTGCTTATGTTACTCAAGAGGATACACTGCTGG
GAACTCTAACACCTAGAGAGACAATTACTTATTCAGCCCATCTTCGACTTCCAACTAG
CATGACGAAAGAAGAGGTAAACGATATTGTGGAGGGAACAATAATGGAAATGGGGCT
AGGGGATTGTGCTGATCGGGTGGTAGGAAATTGGCAAGTAAGAGGAATAAGTGGTGG
CGAGAAAAAGAGACTAAGCATTGCACTTGAAATCCTTGTACGACCTCGTATACTTTTT
CTTGATGAACCTACCACTGGTCTTGACAGTGCCTCAGCATTCTTTGTTGTTCAAGCTC
TTAAAAATATCTCCCGCGATGGAAGAACAGTTATCTCATCCATTCATCAGCCTAGTAGT
GAAGTTTTCGCGTTGTTTGATGACCTCTTTCTGTTGTCTGGTGGAGAGACTGTATATTT
TGGAGAAGCAAAATTGGCAGTACAGTTCTTTGCTGAATCGGGATTTCCATGTCCAAGT
AGGAGAAATCCATCAGATCATTTCCTACGTTGTGTTAATTCAGACTTTGACGTTGTCA
CAGCCACATTGAAAGGCTCACAAAGACTCCGGGAAACACATAAAAGTGACTACTTAA
TGAACATGGCAACAGCAGAAATAAAAGAAATGCTTGCCAGCAAATATAAACGCTCAG
CATATGCAACGAGGGCCAGGTCACGTATGCGAGAACTCTTGGCCACTCAAGGTGTTG
AGATTGAAACAGTAAAAGGAAGTCAAGCTGGTTGGGGAAAGCAACTTTTGACACTG
ACGCGGAGATCATTTGTAAACATGTCAAGAGACAAGGGATATTATTGGTCTCGTATAG
TAATATATATCATTGTAGCTTTTGCCGTTGGTACCCTCTTCTATGATGTTGGCACCAGTT
ACACAGCAATCTTAGCTCGTGGAGCTTGTGGTGGGTTTGTCACGGGCTACATGACTTT
CATGTCCATTGGCGGCTTTCCATCTTTCATAGAAGAAATGAAGGTTTTTACTAAAGAA
AGGCTTAACGGGCACTATGGTGTTGGAGCTTTCATATTGGCAAACTTCCTCTCATCATT
TCCATTCTTAGTTGCAGTTTCACTCATCACTGGGACCATCACTTATTACATGGTGTTTC
GGGCTAGATTCTTCCGTTATGTGTTTTTCTGCTTAAACCTTTTTGGTTGTATCGCTGTCG
TTGAGAGCTGCATGATGATTGTAGCTTCACTTGTTCCAAACTTCTTAATGGGAATCATT
ACAGGAGCTGGTGTGCTTGGAATCATGATGATGACTGCTGGATTTTTCCGTTTGCTGC
CTGATCTTCCCAAGCCAATTTGGCGCTATCCAATTTCATTTATTGGGTATGGGGCATGG
GGTTTACAGGGATCATACAAAAATGACATGATTGGACTTGTATTCGATCCACTAATTCC
TGGCGGAGAGAAACTAAAAGGGGAAGATGTGATCACGAACATGTTCAAATTGTCGTT
GGATCACTCCAAATGGTGGGATTTACTTGCGTTATACTCCCTCATCGTCATGTACAGGC
TTCTATTCTTTATAATTCTCAAGCTAAAAGAGCGAGCTACACCATTTTTCCGATCAATG
TATGCTAAGAGAACGATGCATCAACTCAAAAGGCGTCCTTCATTCAAGAGGAAACCA
TCTTTATCCTATTCAAAAAGGCAACATACTCTCAGGTCATTGTCTTCTCAAGAAGGTCT
TAGTTCTCCAATCCCATAG.
The sequence of the first exon region of the SmABCG2 gene is shown in
SEQ ID NO: 2:
(SEQ ID NO: 2)
ATGGAGATAGAGGTTACAAGTGGTAGTAGTGATATTGAGAAAGGTGTAATGCA
TAGACAAGGAGTTGCATATTTGGTATGGGAAGATTTAACAGTAATGTTGCCAAATTTT
GGTCAGGGACCAACTAAGAAGTTGTTACATGGACTAAGTGGTTATGCTGAACCTGGT
AGAATTATGGCTATTATGGGTCCTTCTGGTTCTGGAAAATCTACACTTCTTGATACTTTA
GCAGGTTAGTCTTTGATTATGAATACTTTGGACTATGTCCGATTAGATAGATGAAAAGA
AATTATCTAGCATTTAAACCCTAGTCGATTGTCAGGAAATAGCCTCTCTACCTCTGTAA
AGTAGTGGTAAGGTCTGCGTACATTTGTGAGATTTCACTGGTATGTTGTTGTTAGTTAA
ATCCTAGTCGA.

The sgRNA sequences for the two target sites are:

Target site 1 (SEQ ID NO: 5):
5′-AAGTTGTTACATGGACTAAG-3′;
Target site 2 (SEQ ID NO: 6):
5′-TCCTCTTCTTCCCATTTAGA-3′.

Primers were designed and synthesized according to the sgRNA sequences for the two target sites, and the sequences were as follows:

KO1F0 (SEQ ID NO: 7):
5′-tgaagttgttacatggactaaggttttagagctagaaatagc-3′;
KO2R0 (SEQ ID NO: 8):
5′-aactcctcttcttcccatttagacaatctcttagtegactctac-3′;
KO1F (SEQ ID NO: 9):
5′-aataatggtctctattgaagttgttacatggactaaggtt-3′;
and
KO2R (SEQ ID NO: 10):
5′-attattggtctctaaactcctcttcttcccatttaga-3′.

### Example 2 Construction of CRISPR/Cas9 Recombinant Vector and Agrobacterium Tumefaciens-Mediated Transformation

First, PCR amplification was performed with a pCBC-DTIT2 intermediate vector as the template and KO1F0 and KO2R0 as the primers to obtain product A1. PCR amplification was then performed with the PCR product A1 as the template and KO1F and KO2R as the primers to obtain product A2. The product was purified and recovered from A2 and the pKSE401 vector were linearized with Bsa I enzyme, respectively, purified, and then ligated overnight at 4° C. using Solution I ligase to obtain pKSE401-G2. The reaction conditions for the above PCR were: pre-denaturation at 95° C. for 5 min, denaturation at 95° C. for 10 sec, annealing at 55° C. for 10 sec, extension at 72° C. for 10 sec, for a total of 35 cycles, and final elongation at 72° C. for 5 min. 10 μL of the ligation product pKSE401-G2 was transformed into Escherichia coli competent cells DH5α, the single clones were obtained, and then colony PCR was performed with 26 pF (SEQ ID NO: 3) and 29pR (SEQ ID NO: 4) as the upstream and downstream primers, wherein the 26 pF was: 5′-TGTCCCAGGATTAGAATGATTAGGC-3′ (SEQ ID NO: 3), and the 29pR was: 5′-AGCCCTCTTCTTTCGATCCATCAA C-3′ (SEQ ID NO: 4). The resulting product having the correct band size was subjected to scale-up culture for 12-16 h. Verification was performed using first-generation sequencing, and the successfully constructed vector with correct sequence was named pKSE401-G2. The plasmid was transformed into Agrobacterium tumefaciens LBA4404, the transformed Agrobacterium tumefaciens LBA4404 was streaked on a YEB solid medium with resistance, and cultured at 28±2° C. in the dark for 2.5 d until single colonies grew. Four single colonies were selected for further identification, wherein the positive strain was the engineered Agrobacterium strain. The strain as cultured in YEB liquid medium (pH 7.2) containing 50 mg/L rifampicin, 500 mg/L streptomycin and 50 mg/L kanamycin at 28±2° C. in the dark at 200 rpm on a shaker for 1.5 d until the bacterial liquid was uniform and the OD600 exceeded 2.0. Seven hundred microliters of bacterial culture and 300 microliters of 50% glycerol were mixed evenly in a 1.5 mL centrifuge tube and cryopreserved at −80° C. for later use.

### Example 3: Genetic Transformation of Eggplant

Preparation of explants: Eggplant seeds were soaked with alcohol having a volume fraction of 75% for 2 min, and rinsed 3 times with sterile water; the seeds were soaked with 1% (V/V) aqueous sodium hypochlorite solution for 10 min, and rinsed 7 times with sterile water; and the seeds were soaked with sterile water for 4 h, sown on an MS solid medium, cultured in a lighting incubator at 26° C. (16 h light)/18° C. (8 h dark) until the cotyledons were flattened, and flattened cotyledons having a length of 6.5 mm were cut to obtain the eggplant explants.

Preparation of engineered Agrobacterium suspension: The strain obtained in Example 2 was inoculated on a resistant YEB solid medium, and cultivated at 28±2° C. in the dark for 2.5 days until single colonies grew. Single colonies were picked and subjected to scale-up culture at 28±2° C. and 200 rpm for 1.5 d using 20 mL of YEB liquid medium at pH 7.2 containing 50 mg/L rifampicin, 500 mg/L streptomycin and 50 mg/L kanamycin. The resulting bacterial culture was inoculated in YEB liquid medium at pH 7.2 containing 50 mg/L rifampicin, 500 mg/L streptomycin and 50 mg/L kanamycin at a volume ratio of 1:100, and subjected to scale-up culture at 28±2° C. and 200 rpm until the OD600 was 2.0. The final culture liquid was centrifuged at 28° C. and 6000 rpm for 8 min. The supernatant was discarded, and the bacterial pellet was washed with a fresh YEB liquid medium once, and then resuspended with 100 mL of MS salt solution to prepare the engineered Agrobacterium suspension.

Preparation of transgenic seedlings: The middle section of the cotyledon was soaked in MS liquid medium for 40 min, and the residual liquid medium on explant was sucked out using sterilized filter paper. The soaked explant was pre-cultured at 26° C. (16 h light)/18° C. (8 h in dark) for 1 day using MS solid medium (pre-culture medium) at pH 5.8 containing sucrose with a mass fraction of 3%, agar with a mass fraction of 0.8%, 1 mg/L indoleacetic acid and 1.7 mg/L zeatin, then immersed into the engineered Agrobacterium suspension for 15 min, then placed back to the original pre-culture medium, co-cultured at 25±2° C. in the dark for 48 h, finally transferred to an MS solid medium (screening medium) with pH 5.8 containing sucrose with a mass fraction of 3%, agar with a mass fraction of 0.8, 1.0 mg/L indoleacetic acid, 1.75 mg/L zein, 500 mg/L carbenicillin and 50 mg/L kanamycin, and cultured at 25±2° C. under a photoperiod of 16 h/d and a light intensity of 1500 lx until calli and buds with resistance grew. Buds having a length of 3 cm were cut off and transferred to an MS solid medium (rooting medium) at pH 5.8 containing sucrose with a mass fraction of 3%, agar with a mass fraction of 0.8%, 250 mg/L carbenicillin and 50 mg/L kanamycin, and cultured at 25±2° C. under a photoperiod of 16 h/d and a light intensity of 1500 lx until rooting. After the rooting, the plants were all subjected to final planting in the heliogreenhouse (32°59′6.17″ N, 114°02′20.13″ E) at the trial base of the Zhumadian Academy of Agricultural Sciences (Typical northern semi-arid region of China).

### Example 4 Identification of Transgenic Plants and Detection of Genome Editing

(1) Identification of transgenic plants: Leaf genomic DNA of the regenerated plant and wild-type eggplant material was extracted, and PCR amplification was performed with a primer pair consisting of Cas9-F and Cas9-R. PCR reaction conditions were: pre-denaturation at 95° C. for 5 min, denaturation at 95° C. for 10 sec, annealing at 55° C. for 30 sec, extension at 72° C. for 1 min 10 sec, for a total of 35 cycles, and final extension at 72° C. for 5 min. Amplification was performed with the following specific primers designed according to Cas9 in vector pKSE401:

Cas9-F (SEQ ID NO: 11):
5′-ACGACGATGACCTGGATAAC-3′;
Cas9-R (SEQ ID NO: 12):
5′-ACCTTGTCATCGAAGAGATGG-3′.

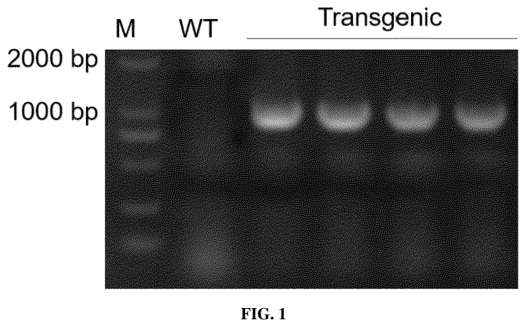

The above PCR amplification products were subjected to 1% agarose gel electrophoresis. The results are shown in FIG. 1. FIG. 1 shows that the PCR amplification product obtained using genomic DNA of wild-type eggplant as the template has no specific band in the agarose gel electrophoresis assay, whereas the PCR amplification product obtained using genomic DNA of the T₀ generation plant as the template shows a specific band with a band size of 1129 bp in the agarose gel electrophoresis assay, indicating that the T₀ generation plant has a Cas9 gene fragment.

### (2) Detection of Genome Editing:

The genomic sequences near the two target sites of LBA4404 were used to design the primer pairs G2-F1/G2-R1 and G2-F2/G2-R2 which were used to detect the editing profile at target1 and target2, respectively.

G2-F1 (SEQ ID NO: 13):
5′-TAGTGATATTGAGAAAGGTG-3′;
G2-R1 (SEQ ID NO: 14):
5′-GAATAGAAGAAATGGACC-3′;
G2-F2 (SEQ ID NO: 15):
5′-TTCAATGAGTAATTTGCCTCTG-3′;
G2-R2 (SEQ ID NO: 16):
5′-CACCCGATCAGCACAATC-3′.

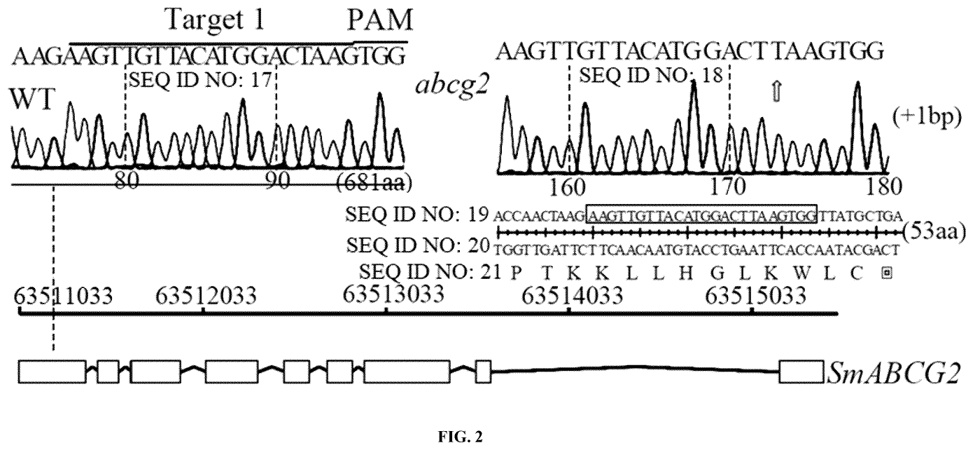

High-fidelity PCR amplification was performed by using the genomic DNA of the transgenic plant obtained in the above step as a template. PCR reaction conditions were: pre-denaturation at 95° C. for 5 min, denaturation at 95° C. for 10 sec, annealing at 55° C. for 10 sec, extension at 72° C. for 10 sec, a total of 32 cycles, and final extension at 72° C. for 5 min. Sequencing was then performed using G2-F1 and G2-F2 primers to detect variations at the target sites. The results show that the editing efficiency of the sgRNA shown in SEQ ID NO: 5 was much higher than that of the sgRNA shown in SEQ ID NO: 6. Compared to wild-type plants, regenerated plants abcg2-1, abcg2-3 and abcg2-4 all have a variation in the sequence at the gene editing site Target1 (FIG. 2), but no mutation at Target2, wherein the type of the variation in the plant is homozygous; and both strands of DNA have an increase of 1 bp at the same site, resulting in premature termination of translation. After planting and screening, a total of 3 mutant lines abcg2-1, abcg2-3 and abcg2-4 were obtained, all of which had serious pollen abortion.

### Example 5: Detection of Pollen Activity

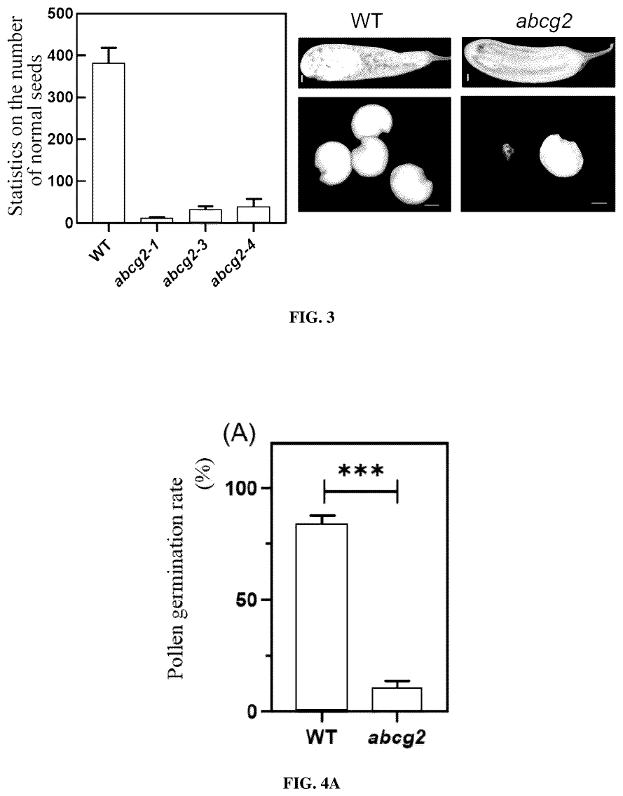

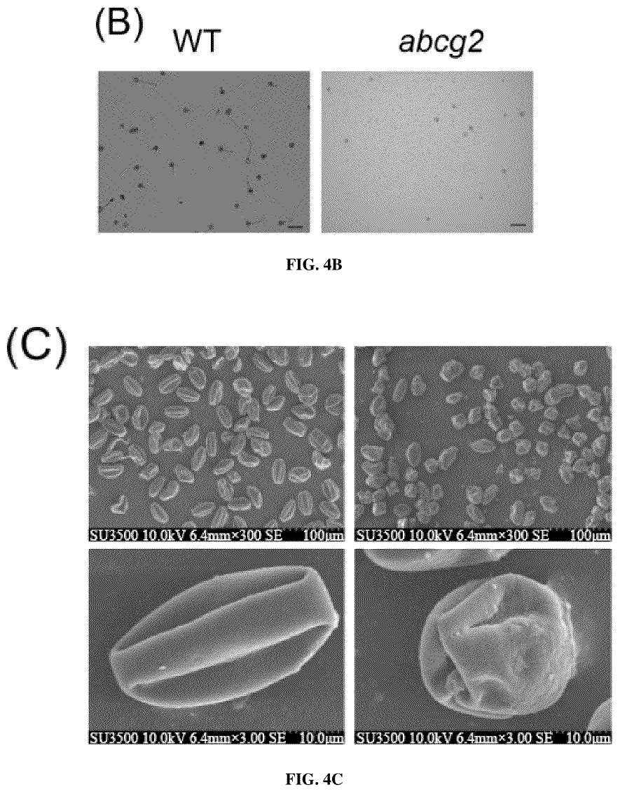

The fertility phenotype of the mutant lines was measured, using “Sanyueqie” as a control. The results are shown in FIG. 3. Compared to the control group, most of the seeds of the abcg2 mutant produced by genome editing had serious abortion. When observed with an optical microscope, it was found that the abcg2 mutant had a significant decrease in pollen germination rate compared to WT (FIG. 4A). In addition, when observed using a scanning electron microscope (HITACHI, Japan), it was found that the pollen of the abcg2 mutant had a serious crumple (FIG. 4C). The results showed that the SmABCG2 gene deletion could cause significant male sterility in eggplant when cultivated in the northern semi-arid region.

The above-mentioned examples are merely intended to describe specific embodiments of the present disclosure, but not to limit the scope of the present disclosure. Various alterations and improvements made to the technical solutions of the present disclosure by those of ordinary skill in the art without departing from the spirit of the present disclosure all fall within the scopes of protection defined in the claims of the present disclosure.

What is claimed is:

1. A method for site-directed mutagenesis of a gene in eggplant, comprising:
transforming an eggplant with a recombinant Agrobacterium tumefaciens comprising the sgRNA of SEQ ID NO:5 or the sgRNA combination of SEQ ID NO:5 and SEQ ID NO:6-G2.

2. The method of claim 1, wherein said recombinant Agrobacterium tumefaciens comprises pKSE401-G1.

3. The method of claim 1, wherein said recombinant Agrobacterium tumefaciens comprises pKSE401-G2.

4. The method of claim 1, further comprising the step of screening the transformed eggplant for plants having an SmABCG2 gene knock-out.

5. The method of claim 4, wherein the SmABCG2 gene knock-out line of eggplant is a male sterile plant.

6. A CRISPR/Cas9 editing system, comprising an sgRNA or an sgRNA combination, wherein said sgRNA has the sequence of SEQ ID NO: 5 and said sgRNA combination further an sgRNA of SEQ ID NO: 6.

7. The editing system of claim 6, comprising an sgRNA of SEQ ID NO: 5.

8. The editing system of claim 7, further comprising an sgRNA of SEQ ID NO:6.

9. The editing system of claim 7, comprising pKSE401-G1.

10. The editing system of claim 8, comprising pKSE401-G2.

11. A recombinant eggplant comprising a recombinant Agrobacterium tumefaciens wherein said A. tumefaciens comprises the sgRNA of SEQ ID NO:5 or the sgRNA combination of SEQ ID NO: 5 and SEQ ID NO:6-G2.

12. The recombinant eggplant of claim 11, wherein said recombinant Agrobacterium tumefaciens comprises pKSE401-G1.

13. The recombinant eggplant of claim 11, wherein said recombinant Agrobacterium tumefaciens comprises pKSE401-G2.

14. The recombinant eggplant of claim 11, wherein said eggplant has an SmABCG2 gene knock-out.

15. The recombinant eggplant of claim 14, wherein said eggplant is a male sterile plant.
